(19) United States
(12) Patent Application Publication
(10) Pub. No.: US 2017/0165027 A1
(43) Pub. Date: Jun. 15, 2017

(54) Autonomous Light Tracking Device and Method to Facilitate Medical Procedures

(71) Applicant: LuceLine Unlimited, Inc., Wayzata, MN (US)

(72) Inventor: Stephen D. Paulson, Orono, MN (US)

(73) Assignee: LuceLine Unlimited, Inc., Wayzata, MN (US)

(21) Appl. No.: 15/215,407

(22) Filed: Jul. 20, 2016

Related U.S. Application Data
(60) Provisional application No. 62/179,911, filed on May 21, 2015.

(51) Int. Cl. A61B 90/35; G01J 1/02; F21V 21/28; G01J 1/44; F21V 14/02; F21V 21/15

(52) U.S. Cl. CPC A61B 90/35; F21V 14/02; F21V 21/15; F21V 21/28; G01J 1/44; G01J 1/0238; F21W 2131/205

(57) ABSTRACT

The present invention describes an autonomous light tracking device which hands-off tracks the physician's line-of-sight while examining a patient utilizing infrared targeting and tracking techniques, thereby maintaining sterility of the examination by eliminating the need for the physician to manually adjust the overhead lighting to better view the patient.

### CROSS-REFERENCE TO RELATED APPLICATIONS

This application claims the benefit of U.S. Provisional Patent Application No. 62/179,911 filed May 21, 2015,which hereby is incorporated herein in its entirety by reference thereto.

### FIELD OF THE INVENTION

The present invention is directed generally to medical devices and more specifically to an autonomous light tracking device which hands-off tracks the physician's line-of-sight while examining a patient utilizing infrared targeting and tracking techniques, thereby maintaining sterility of the examination by eliminating the need for the physician to manually adjust the overhead lighting to better view the patient.

### BACKGROUND

Autonomous tracking of infrared energy is employed in many of the military aircrafts currently in service. Anyone who has watched an aerial recording of a bombing mission and viewed the cross hairs overlaying a target just prior to the explosion has viewed first hand an airborne autonomous tracking of a target. In a military mission, the target is typically illuminated by an airborne infrared laser, and an infrared detector employed as part of the bomb or missile tracks the infrared energy reflected by the target. The bomb or missile has onboard electronics which guides the bomb/missile to the source of the reflected infrared energy ultimately reaching a direct impact with the target. In addition, autonomous infrared tracking systems have been developed to track airborne targets, both for military and non-military applications. Not surprisingly, over the years a series of technical inventions can be found in US patents and patent applications which have enabled this technology.

Examples of the diversity of such prior art can be found in the following:

US Patent Application Publication No. 2010/0283988 published Nov. 11, 2010 entitled “Semi-Active Optical Tracking System” which describes a method and device for tracking an airborne target via illumination by an infrared laser diode source and means to filter out airborne artifact signatures from clouds, smoke, and other artifacts to track the intended target.

US Patent Application Publication No. 2011/0142284 published Jun. 16, 2011 entitled “Method and Apparatus for Acquiring Accurate Background Infrared Signature Data on Moving Targets” which describes a particular hardware design applicable to military applications for tracking an airborne target utilizing an infrared targeting/tracking methodology.

The infrared tracking technology has also made its way into the sporting world as represented in the following invention:

US Patent Application Publication No. 2010/0026809 published Feb. 4, 2010 entitled “Camera-Based Tracking and Position Determination for Sporting Events” which discloses a system and method for camera based infrared tracking of sporting events such as tracking the flight of a golf ball or the movement of sports athletes.

The present invention discloses a device and method to further advance this technology into medical technology as described herein.

### SUMMARY OF THE INVENTION

The present invention is directed generally to medical devices and more specifically to a method to autonomously control lighting devices used during medical procedures.

One embodiment of the present invention describes an autonomous light tracking device to facilitate a medical procedure comprising a source of infrared energy attached to the temple region of a physician performing the medical procedure, the source of infrared energy collimated in a pencil-like beam and oriented parallel to the physician's line of sight, a lighting device configured with an infrared energy detector incorporating an appropriate filter to only respond to the infrared source attached to the physician, the output of the lighting device infrared energy detector in electrical communication with a control unit embedded within said lighting device, the lighting device in mechanical communication with a support structure and attached thereto by an articulated arm, the articulated arm motor driven and capable of rotation about 3 orthogonal axes, and the control unit capable of autonomously scanning the motor driven articulated arm to maximize infrared energy received by the infrared detector.

The above summary of the present invention is not intended to describe each illustrated embodiment or every implementation of the present invention. The figures and the detailed description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1A shows one embodiment of the present invention depicting a physician initially observing the leg region of a patient in a medical exam room wherein the autonomously controlled lighting device has scanned and is now illuminating the same leg region the physician is viewing.

FIG. 1B shows the same medical exam room after the physician has changed his field of view to the chest region of the patient, wherein the autonomously controlled lighting device has re-scanned and is now illuminating the same chest region the physician is viewing.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

The present invention is directed generally to medical devices and more specifically to a method to autonomously control lighting devices used during medical procedures.

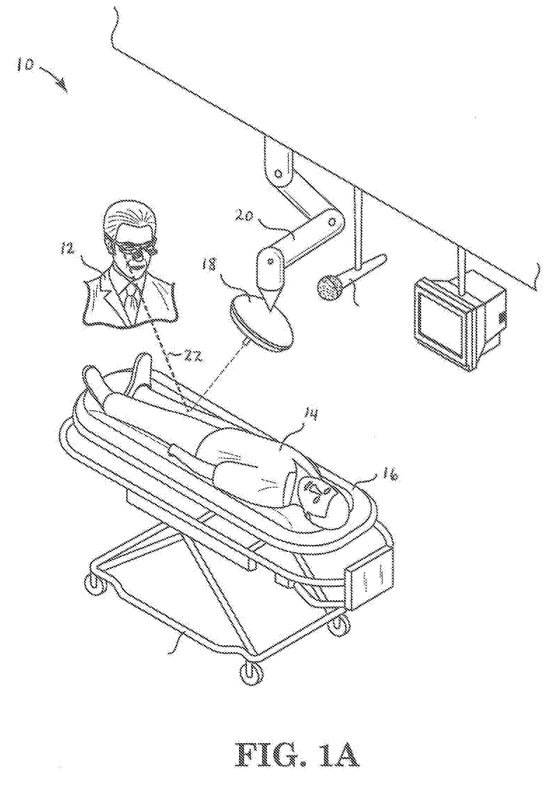

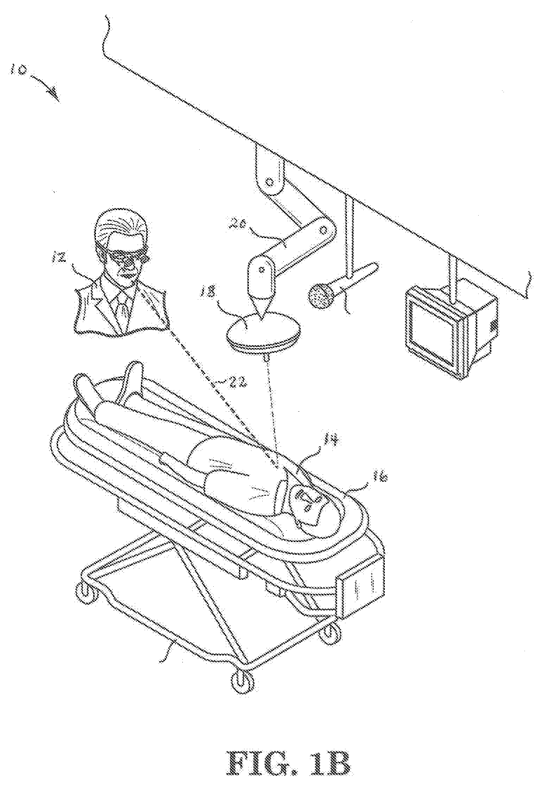

One embodiment of the present invention is depicted in FIG. 1A.

FIG. 1A shows a medical exam room 10 with a physician 12 viewing a patient 14 who is lying on a typical examination table 16. The exam room 10 is shown with an overhead lighting device 18 which may be attached to a motor driven articulated arm 20, which in turn may be attached to the ceiling of the exam room 10 or any appropriate overhead support member.

As shown in FIG. 1A, the physician 12 may initially be viewing the leg region of the patient 14. The physician 14 is wearing an optical viewing device with a source of infrared energy attached thereto, near the physician's temple region. The infrared source of energy may be collimated in a pencil-like beam 22 as shown in FIG. 1A and oriented in parallel with the physician's line-of-sight. In this configuration, the infrared beam may be incident on the patient's leg proximate to the area the physician is viewing with his unaided eye.

The lighting device 18 may be configured with an infrared energy detector incorporating an appropriate filter to only respond to the infrared energy emanating from the source attached to the said physician. In addition, the output of the lighting device's infrared energy detector may be in electrical communication with a control unit embedded within the lighting device 18. The control unit (not shown explicitly in FIG. 1A) may incorporate programmable logic circuitry (PLC) and may be programmed to output executable commands to the motors which actuate movement of the articulated arm 20. In one embodiment of the invention, the programmable logic circuitry may be pre-programmed to output a command to the articulated arm 20 to scan the lighting device 18 over a 2 dimensional region corresponding to where a patient 14 may typically be located. In this embodiment of the present invention, the programmable logic circuitry may also be pre-programmed to cease the 2D scanning mentioned above when the received infrared energy outputted from the infrared detector reaches a predetermined threshold and dwells on that location.

FIG. 1A depicts the scenario wherein the programmable logic circuitry has sequentially scanned the patient and has ceased scanning and has the lighting device 18 fixed on the location where the infrared source is incident 22 upon the patient's leg region.

FIG. 1B depicts the scenario wherein the physician 12 has altered his line-of-sight from the patient's leg region to the patient's chest region. When the physician first changes his line-of-sight away from the patient's leg region, the lighting device's infrared detector would lose the infrared signal that was causing the programmable logic circuitry to lock the articulated arm in place, and the programmable logic circuitry would issue the command to the articulated arm to begin a 2D scan in search of the new infrared energy “hot spot” on the patient. Eventually, the scan would autonomously locate and fixate on the new location of the infrared energy incident on the patient's chest thereby illuminating that area with visible light to aid the physician in his examination.

The present invention should not be considered limited to the particular examples described above, but rather should be understood to cover all aspects of the invention as fairly set out in the attached claims. Various modifications to the shape and form factors described above, equivalent processes to which the present invention may be applicable will be readily apparent to those of skill in the art to which the present invention is directed upon review of the present specification. For example, the present invention anticipates that the following claims are intended to cover such modifications and devices.

I claim:

1. An autonomous light tracking device to facilitate a medical procedure comprising:
a source of infrared energy attached to the temple region of a physician performing the said medical procedure;
said source of infrared energy collimated in a pencil-like beam and oriented parallel to the physician's line of sight;
a lighting device configured with an infrared energy detector incorporating an appropriate filter to only respond to the infrared source attached to the said physician;
output of said lighting device infrared energy detector in electrical communication with a control unit embedded within said lighting device;
said lighting device in mechanical communication with a support structure and attached thereto by an articulated arm;
said articulated arm motor driven and capable of rotation about 3 orthogonal axes; and
said control unit capable of autonomously scanning the said motor driven articulated arm to maximize infrared energy received by the said infrared detector.

2. The device of claim 1 wherein the source of infrared energy may be a laser diode.

3. The device of claim 1 wherein the wherein the source of infrared energy may be an infrared light emitting diode.

4. The energy detector of claim 1 wherein the detector surface may be partitioned into four quadrants.

5. The control unit of claim 1 incorporating a programmable logic circuit.

6. The control unit of claim 5 wherein the programmable logic circuit has been programmed to scan the articulated arm until the infrared energy received by each of the four quadrants has reached a predetermined level.
